(19) United States
(12) Patent Application Publication
(10) Pub. No.: US 2024/0118403 A1
(43) Pub. Date: Apr. 11, 2024

(54) OCCUPANT DETECTION DEVICE AND OCCUPANT DETECTION METHOD

(71) Applicant: Mitsubishi Electric Corporation, Tokyo (JP)

(72) Inventors: Naoya BABA, Tokyo (JP); Takumi TAKEI, Tokyo (JP)

(73) Assignee: Mitsubishi Electric Corporation, Tokyo (JP)

(21) Appl. No.: 18/273,443

(22) PCT Filed: Feb. 24, 2021
(86) PCT No.: PCT/JP2021/006719

§ 371 Date: Jul. 20, 2023

(51) Int. Cl. G01S 13/42; B60N 2/00; G01C 19/00

(52) U.S. Cl. CPC G01S 13/42; B60N 2/0024; G01C 19/00; B60N 2220/00

(57) ABSTRACT

Disclosed is an occupant detection device that detects an occupant riding in a vehicle, including: a first occupant detection unit to output at least one detection result about the occupant, the detection result being detected using an electric wave, each of the at least one detection result being about a corresponding one of detection result types; a degree of influence determination unit to acquire the vibration amount of the vehicle, and to use the vibration amount and the detection result from the first occupant detection unit, to thereby determine, as to each of the detection result types, a degree of influence which depends on the vibration amount; and an output determination unit to determine, using the degree of influence, whether to adopt or reject the detection result, and to, when determining to adopt the detection result, cause the detection result to be outputted.

### TECHNICAL FIELD

The present disclosure relates to an occupant detection device for and an occupant detection method of detecting an occupant riding in a vehicle.

### BACKGROUND ART

Conventionally, in a technique of detecting an occupant riding in a vehicle using an electric wave sensor, there is a case in which erroneous detection occurs because of vibrations.

To cope with this, Patent Literature 1 discloses an occupant detection device that prevents erroneous detection caused by vibrations.

The occupant detection device of Patent Literature 1 estimates that, when the vehicle speed V of the vehicle becomes greater than or equal to a vehicle speed threshold Vth, a vibration occurs in each occupant as the vehicle travels, and raises an intensity threshold Sth used for detecting an occupant to an intensity threshold Sth′, thereby reducing the sensitivity of the detection using an electric wave and preventing erroneous detection.

### CITATION LIST

### Patent Literature

Patent Literature 1: JP 2017-181225 A

### SUMMARY OF INVENTION

### Technical Problem

However, a problem with the occupant detection device of Patent Literature 1 is that an occupant detection result using an electric wave cannot be outputted in a state where vibrations occur. Accordingly, there is a case in which it is impossible to output an error-free occupant detection result even if multiple types of occupant detection provided by an electric wave sensor contain a type of occupant detection whose result is not affected by vibrations in a state where the vibrations occur.

It is an object of the present disclosure to provide an occupant detection device that can output an occupant detection result with few errors even in a state where vibrations occur.

### Solution to Problem

An occupant detection device of the present disclosure detects an occupant riding in a vehicle, and includes: a first occupant detection unit to output at least one detection result about the occupant, each of the at least one detection result being detected using an electric wave, each of the at least one detection result being about a corresponding one of detection result types; a degree of influence determination unit to acquire a vibration amount of the vehicle, and to use the vibration amount and the at least one detection result from the first occupant detection unit, to thereby determine, as to each of the detection result types, a degree of influence which depends on the vibration amount; and an output determination unit to determine, using the degree of influence, whether to adopt or reject a corresponding one of the at least one detection result, and to, when determining to adopt the corresponding one of the at least one detection result, cause the corresponding one of the at least one detection result to be outputted.

### Advantageous Effects of Invention

According to the present disclosure, because the occupant detection device is configured as above, there is provided an advantage of being able to provide an occupant detection device that outputs an occupant detection result with few errors even in a state where vibrations occur.

### BRIEF DESCRIPTION OF DRAWINGS

FIG. 1 is a diagram showing the configuration of an occupant detection system including an occupant detection device according to Embodiment 1;

FIG. 2 is a view explaining an example of a detection method which is based on an electric wave sensor in the occupant detection system;

FIG. 3 is a table explaining an example of pieces of information contained in occupant detection results in an occupant detection unit of the occupant detection device;

FIG. 4 is a table explaining an example of a degree of vibration influence for each of the types of the occupant detection results, and a corrected vibration amount after correction which is corrected using the degree of the influence;

FIG. 5 shows tables explaining an example of correcting degrees of reliability of occupant detection results using degrees of vibration influence;

FIGS. 6A and 6B are diagrams each showing an example of the hardware configuration of the occupant detection system including the occupant detection device;

FIG. 7 is a flowchart showing the processing performed in the occupant detection device;

FIG. 8 is a flowchart showing a detailed example of the processing performed in the occupant detection device according to Embodiment 1;

FIG. 9 is a diagram showing the configuration of an occupant detection system including an occupant detection device according to Embodiment 2;

FIGS. 10A and 10B are diagrams each showing an example of the hardware configuration of the occupant detection system including the occupant detection device according to Embodiment 2; and

FIG. 11 is a flowchart showing a detailed example of the processing performed in the occupant detection device according to Embodiment 2.

### DESCRIPTION OF EMBODIMENTS

Hereinafter, in order to explain the present disclosure in greater detail, embodiments of the present disclosure will be explained with reference to the accompanying drawings.

### Embodiment 1

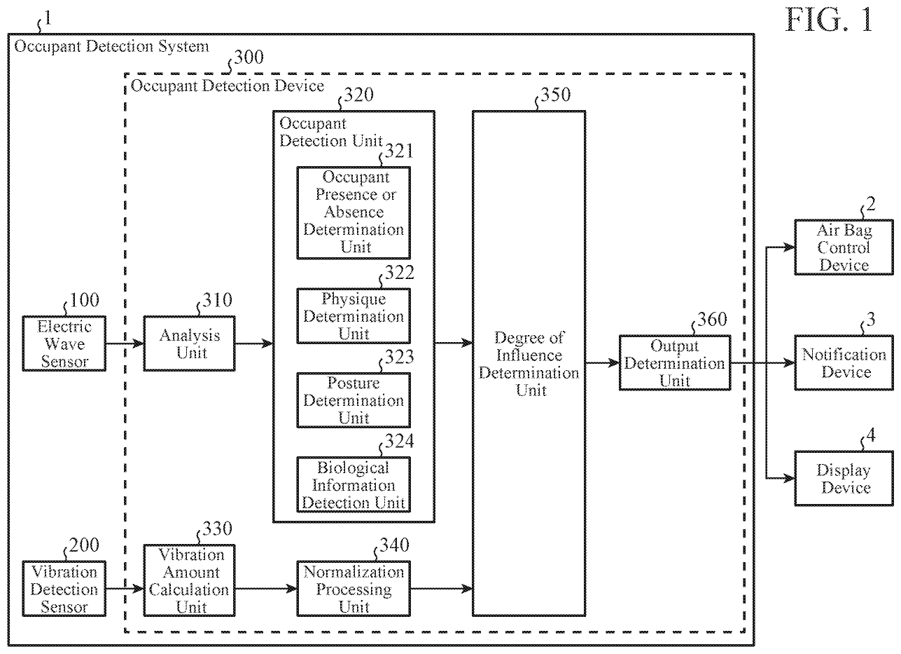

An occupant detection device according to Embodiment 1 and an occupant detection system including this occupant detection device will be explained using FIGS. 1 to 8.

FIG. 1 is a diagram showing the configuration of the occupant detection system 1 including the occupant detection device 300 according to Embodiment 1.

The occupant detection system 1 detects an occupant riding in a vehicle and outputs a detection result to pieces of vehicle-mounted equipment. The occupant should just be one of living things including human beings and animals.

The occupant detection system 1 and each of the pieces of vehicle-mounted equipment are connected in such a way as to be able to communicate with each other.

The pieces of vehicle-mounted equipment are, for example, an air bag control device 2, a notification device 3, and a display device 4. When acquiring a detection result from the occupant detection system 1, the air bag control device 2 controls an airbag using the detection result.

When acquiring a detection result from the occupant detection system 1, the notification device 3 generates information to be notified to an occupant using the detection result, and notifies the occupant of the information.

When acquiring a detection result from the occupant detection system 1, the display device 4 generates information to be displayed using the detection result, and displays the information.

In the present disclosure, the pieces of vehicle-mounted equipment should just acquire a detection result from the occupant detection system 1 and use the detection result for processing, and are not limited to the above-mentioned examples.

The occupant detection system 1 shown in FIG. 1 includes an electric wave sensor 100, a vibration detection sensor 200, and the occupant detection device 300.

The electric wave sensor 100 transmits and receives an electric wave.

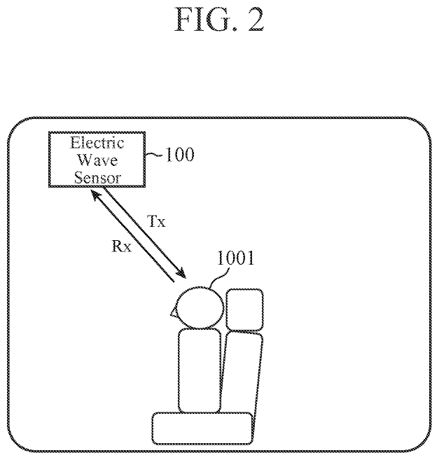

FIG. 2 is a view explaining an example of a detection method which is based on the electric wave sensor 100 in the occupant detection system 1.

The electric wave sensor 100 is mounted, for example, close to the ceiling of the vehicle, as shown in FIG. 2. The electric wave sensor 100 outputs, to an analysis unit 310 of the occupant detection device 300, both signals of a transmission wave Tx which is transmitted toward a seat in the vehicle cabin, and a received wave Rx which is caused by the reflection of the transmission wave Tx by a detection target 1001. The position of the electric wave sensor 100 is not limited to the installation position in the example shown in FIG. 2, and the electric wave sensor 100 should just be disposed at a position where the electric wave sensor 100 can appropriately transmit the transmission wave Tx toward the detection target 1001 and receive the received wave Rx from the detection target 1001.

In the present disclosure, the electric wave sensor 100 is also referred to as a first sensor that uses an electric wave.

The vibration detection sensor 200 shown in FIG. 1 includes, for example, a gyro sensor, and detects rotation angular speeds per unit time in three axes: a pitch axis, a roll axis, and a yaw axis. For example, when the vehicle travels a bumpy road, the angular speed in the pitch axis which is a forward and backward rotation becomes large. Using this angular speed, a vibration amount calculation unit 330 which will be mentioned later can calculate a vibration amount.

The vibration detection sensor 200 is not limited to a gyro sensor, and should just be a sensor that can acquire a signal which makes it possible to detect or estimate vibrations of the vehicle, such as a signal indicating acceleration detected by an acceleration sensor, a signal indicating the speed of the vehicle detected by an acceleration sensor, or a signal indicating a pressure change detected by a seating pressure sensor.

The occupant detection device 300 shown in FIG. 1 receives the signals outputted from the electric wave sensor 100 and the signal outputted from the vibration detection sensor 200, and outputs detection results (also referred to as “occupant detection results” hereinafter) including the presence or absence of an occupant, the physique of the occupant, the posture of the occupant, and biological information about the occupant to the pieces of vehicle-mounted equipment described above.

The occupant detection device 300 includes the analysis unit 310, an occupant detection unit 320, the vibration amount calculation unit 330, a normalization processing unit 340, a degree of influence determination unit 350, and an output determination unit 360. The occupant detection device 300 also includes a not-illustrated control unit.

The analysis unit 310, the occupant detection unit 320, the vibration amount calculation unit 330, the normalization processing unit 340, the degree of influence determination unit 350, and the output determination unit 360, which are included in the occupant detection device 300, may be distributed among servers on a network.

The analysis unit 310 calculates the distance to the detection target 1001, a speed and an angle with respect to the detection target 1001, etc. on the basis of the signals of the transmission wave Tx and the received wave Rx from the electric wave sensor 100, and outputs them to the occupant detection unit 320. As a more concrete calculation method, a known technique, such as a pulse method or a frequency modelated continous wave (FMCW) method, should just be used. Therefore, a detailed explanation of the calculation method is omitted here.

The occupant detection unit 320 outputs the occupant detection results about an occupant, each of the occupant detection results being detected using an electric wave, each of the occupant detection results being about a corresponding one of occupant detection result types.

In the present disclosure, the occupant detection unit 320 is also referred to as a first occupant detection unit. Further, in the present disclosure, the occupant detection results provided by the first occupant detection unit are also referred to as first detection results.

Concretely, the occupant detection unit 320 acquires analysis results which are based on the signals from the electric wave sensor 100 from the analysis unit 310, and performs detection about an occupant. The occupant detection results are, for example, multiple types of detection results including the presence or absence of an occupant of each seat, the physique of the occupant, the posture of the occupant, and biological information about the occupant. Each of the occupant detection results includes, in addition to information indicating the detection result itself, information for identifying the type of the occupant detection result, and a degree of reliability corresponding to the occupant detection result.

In addition, concretely, the occupant detection unit 320 includes at least one of an occupant presence or absence determination unit 321, a physique determination unit 322, a posture determination unit 323, and a biological information detection unit 324.

The occupant presence or absence determination unit 321 expresses a movable body (occupant) with three-dimensional (horizontal, vertical, and depth directions) information on the basis of the information about the distance to the detection target 1001 and the information about the speed with respect to the detection target 1001, which are acquired from the analysis unit 310, and determines the presence or absence of the occupant from the three-dimensional information. The occupant presence or absence determination unit 321 outputs, as an occupant detection result, information for identifying a seat position, occupant presence or absence information indicating the presence or absence of the occupant, and the degree of reliability of the detection result. The occupant presence or absence information is, for example, binary information indicating presence or absence.

As a method of determining the presence or absence of an occupant, there is, for example, a method of preparing a statistical model for occupant presence or absence in advance, and calculating a determination result and the degree of reliability of the determination result from the degree of similarity between detected three-dimensional information and the statistical model. Further, a rule for judging the result may be determined and a moving average of output values within a certain time period may be calculated, thereby providing a degree of reliability.

The physique determination unit 322 expresses a movable body (occupant) with three-dimensional (horizontal, vertical, and depth directions) information on the basis of the information about the distance to the detection target 1001 and the information about the speed with respect to the detection target 1001, which are acquired from the analysis unit 310, and determines the physique of the occupant from the three-dimensional information. The physique determination unit 322 outputs, as an occupant detection result, information for identifying a seat position, physique information indicating the physique of the occupant, and the degree of reliability of the occupant detection result. The physique information is, for example, information indicating a result of classifying the physique of the occupant as one of adult, child and so on.

As a method of determining the physique of an occupant, there is, for example, a method of preparing statistical models for physiques (adult, child, and so on) in advance, and calculating a determination result and the degree of reliability of the determination result from the degrees of similarity between detected three-dimensional information and the statistical models. Further, a rule for judging the result may be determined and a moving average of output values within a certain time period may be calculated, thereby providing a degree of reliability.

The posture determination unit 323 expresses a movable body (occupant) with three-dimensional (horizontal, vertical, and depth directions) information on the basis of the information about the distance to the detection target 1001 and the information about the speed with respect to the detection target 1001, which are acquired from the analysis unit 310, and determines the posture of the occupant from the three-dimensional information. The posture determination unit 323 outputs, as an occupant detection result, information for identifying a seat position, posture information indicating the posture of the occupant, and the degree of reliability of the occupant detection result. The posture information is, for example, information indicating a result of classifying the posture as one of predetermined occupant posture patterns, such as normality, falling sideways, and facedown.

As a method of determining the posture of an occupant, there is, for example, a method of preparing statistical models for postures (normality, facedown, and so on) in advance, and calculating a determination result and the degree of reliability of the determination result from the degrees of similarity between detected three-dimensional information and the statistical models. Further, a rule for judging the result may be determined and a moving average of output values within a certain time period may be calculated, thereby providing a degree of reliability.

The biological information detection unit 324 performs signal processing on a reflection signal containing a variation in an occupant's body surface, for example, thereby separating and extracting a waveform of breathing and a waveform of heart beats, and calculating a breathing rate and a heart rate from the waveforms after the separation. This means that the appearance of a fine body surface variation in the reflection signal acquired from the analysis unit 310 and reflected from the detection target 1001, the variation being caused by the occupant's breathing and heart beats, is used.

A known technique should just be used as the signal processing for extracting breathing and heart beats, and thus a detailed explanation of the technique will be omitted hereinafter.

For example, when a breathing rate and a heart rate are calculated at certain intervals (e.g., at intervals of one second), a moving average of results of success or failure (e.g., 0 or 1) of detection of breathing and heart beats is calculated within a predetermined time period (e.g., a time period of one minute), and the result of the calculation of the moving average is defined as the degree of reliability corresponding to the breathing rate and the heart rate.

The biological information detection unit 324 outputs, as an occupant detection result, information for identifying a seat position, biological information, and the degree of reliability of the occupant detection result. The biological information is, for example, information indicating a breathing rate per minute and a heart rate per minute, or the like.

The biological information detection unit 324 may use the vibration amount of the vehicle acquired from the acceleration sensor or the like, and thereby may be able to distinguish between breathing/heart beats and vibrations at the time of the calculation of the breathing rate and the heart rate.

Each of the calculation methods in the occupant presence or absence determination unit 321, the physique determination unit 322, the posture determination unit 323, and the biological information detection unit 324 which are included in the occupant detection unit 320 may use one of various well-known techniques including a machine learning method of, for example, learning a relation between a pre-defined feature quantity effective for classification and a desired result in advance on the basis of information acquired from the analysis unit 310, and classifying information acquired from the analysis unit 310 according to the learning model.

The occupant detection unit 320 may perform the detection again at predetermined certain intervals or in accordance with situations. The situations include, for example, at least one of a time when the vehicle stops, a time when the vehicle speed becomes less than or equal to a predetermined speed, a time when the vibration amount of the vehicle becomes less than or equal to a predetermined amount, a time when a vehicle door is closed, and a time when a change occurs in the position of an occupant in the vehicle.

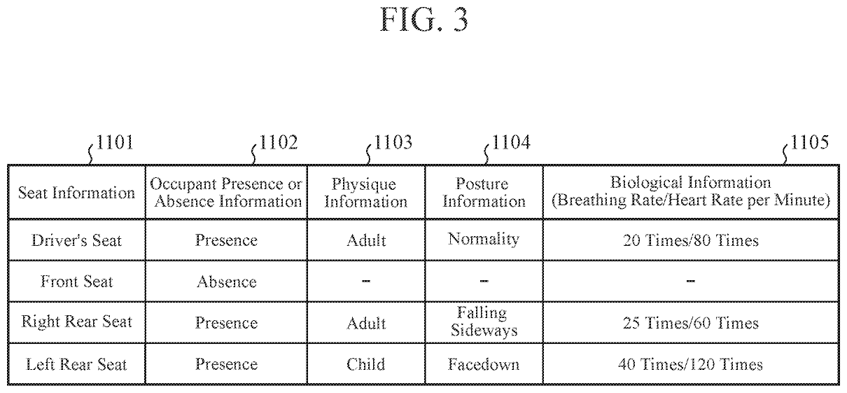

FIG. 3 is a table explaining an example of the pieces of information contained in the occupant detection results in the occupant detection unit 320 of the occupant detection device 300.

The seat information 1101 for identifying a seat position is, for example, information such as the driver's seat, a front seat, a right rear seat, or a left rear seat.

The occupant presence or absence information 1102 is, for example, information indicating the presence or absence of an occupant.

The physique information 1103 is, for example, information such as an adult or a child.

The posture information 1104 is, for example, information such as normality, falling sideways, or facedown.

The biological information 1105 is, for example, a breathing rate and a heart rate per minute, and is information such as 20 times/80 times, times/60 times, or 40 times/120 times.

Further, a degree of reliability is added to each of the following pieces of information: the occupant presence or absence information 1102, the physique information 1103, the posture information 1104, and the biological information 1105. The description of the degree of reliability is omitted in FIG. 3.

The vibration amount calculation unit 330 analyzes the amplitude, the number of vibrations, etc. in the rotation angular speed in each of the three axes: the pitch axis, the roll axis, and the yaw axis, which is acquired from the vibration detection sensor 200, and thereby calculates the vibration amount in each of the axes. As an alternative, the vibration amount calculation unit 330 may calculate the vibration amount using information such as the acceleration, the vehicle speed, the accelerator quantity, the steering angle, the blinker lighting state, or the brake amount. One of various well-known techniques can be used for the calculation of the vibration amount, and a detailed explanation of these techniques will be omitted hereinafter.

In a case where the vehicle speed of the vehicle is used for the vibration detection sensor 200, the vehicle speed itself may be handled as the vibration amount, for example. More specifically, although in Embodiment 1 the configuration of including the vibration amount calculation unit 330 is explained, the occupant detection device 300 of the present disclosure may use information indirectly indicating the vibration amount, and thus does not necessarily include the vibration amount calculation unit 330.

The normalization processing unit 340 normalizes the vibration amount acquired from the vibration amount calculation unit 330. As a normalizing method, for example, a method of setting the vibration amount to “1” when vibrations do not occur at all, and normalizing the vibration amount in such a way as to cause the vibration amount to approach “0” as vibrations become large should just be used. The normalization processing unit 340 outputs the vibration amount normalized thereby (referred to as the “normalized vibration amount” hereinafter) to the degree of influence determination unit 350.

Although in Embodiment 1 the normalized vibration amount which is acquired by normalizing the vibration amount is used, the occupant detection device 300 of the present disclosure should just be able to determine, as to each of the types of the occupant detection results, whether or not to output the occupant detection result in consideration of the influence which depends on the vibration amount, and does not necessarily include the normalization processing unit 340.

The degree of influence determination unit 350 acquires the normalized vibration amount from the normalization processing unit 340, and uses this normalized vibration amount and the occupant detection results (first detection results) from the occupant detection unit 320, to determine the degree of influence of vibrations as to each of the types of the occupant detection results.

For example, concretely, from a degree of influence on vibrations (referred to as the “degree of vibration influence” hereinafter, where the degree of vibration influence is defined in advance as to each of the types of the occupant detection results), the degree of influence corresponding to an occupant detection result type, and the normalized vibration amount acquired from the normalization processing unit 340, the degree of influence determination unit 350 calculates a vibration amount (referred to as a “corrected vibration amount” hereinafter) which is acquired by correcting the normalized vibration amount with the value of the degree of vibration influence.

Although the “corrected vibration amount” is used in the explanation, a term “degree of influence” can be simply used in the present disclosure instead of the term “corrected vibration amount” because the “corrected vibration amount” is a value which is acquired by correcting the normalized vibration amount with the value of the degree of vibration influence. More specifically, the degree of influence determination unit 350 calculates, as to each of the types of the occupant detection results (first detection results), the “corrected vibration amount” as the degree of influence which depends on the vibration amount.

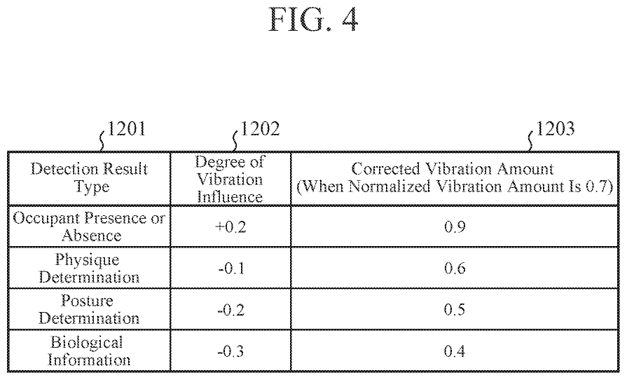

FIG. 4 is a table explaining an example of the degree of vibration influence for each of the types of the occupant detection results, and the corrected vibration amount after correction which is corrected using the degree of the influence.

For example, as shown in FIG. 4, the degree of vibration influence 1202 is determined in advance for each of the types 1201 of the occupant detection results, and the degree of influence determination unit 350 calculates the corrected vibration amount 1203 as to each of the types of the occupant detection results.

The corrected vibration amounts 1203 corresponding to the respective detection types 1201 when the normalized vibration amount is “0.7” are shown in FIG. 4. When the type 1201 of a detection result is “occupant presence or absence”, the degree of influence determination unit 350 adds the degree of vibration influence 1202 of “+0.2” to the normalized vibration amount of “0.7”, thereby calculating the corrected vibration amount 1203 of “0.9.” When the type of a detection result is “physique determination”, the degree of influence determination unit 350 adds the degree of vibration influence 1202 of “−0.1” to the normalized vibration amount of “0.7”, thereby calculating the corrected vibration amount 1203 of “0.6.” When the type 1201 of a detection result is “posture determination”, the degree of influence determination unit 350 adds the degree of vibration influence 1202 of “−0.2” to the normalized vibration amount of “0.7”, thereby calculating the corrected vibration amount 1203 of “0.5.” When the type 1201 of a detection result is “biological information”, the degree of influence determination unit 350 adds the degree of vibration influence 1202 of “−0.3” to the normalized vibration amount of “0.7”, thereby calculating the corrected vibration amount 1203 of “0.4.” The “degrees of vibration influence” in FIG. 4 are determined in such a way that their values increase in the negative direction as the influence of vibrations on the detection results becomes stronger, while their values increase in the positive direction as the influence of vibrations on the detection results becomes weaker.

In this example, although the corrected vibration amounts 1203 do not exceed the maximum “1” of the normalized vibration amount, the corrected vibration amount 1203 in the detection result type 1201 of “occupant presence or absence” is “1.1” and thus exceeds the maximum “1” of the normalized vibration amount if the normalized vibration amount is “0.9.” In such a case, the degree of influence determination unit 350 performs a process of correcting the corrected vibration amount 1203 of “1.1” to “1.0.”

Although the degree of influence determination unit 350 determines the degree of vibration influence 1202 for each of the types of the occupant detection results in advance, the degree of influence determination unit 350 may determine the degree of vibration influence while also taking into consideration the degree of influence which depends on each seat.

The output determination unit 360 uses the corrected vibration amount (the degree of influence which depends on the vibration amount) which is acquired from both the degree of vibration influence for each of the types of the occupant detection results, and the normalized vibration amount, to determine whether to adopt or reject the occupant detection result (first detection result), and, when determining to adopt the occupant detection result, causes the occupant detection result to be outputted.

For example, the output determination unit 360 determines the necessity or unnecessity of adopting the detection result on the basis of the occupant detection result acquired from the occupant detection unit 320 via the degree of influence determination unit 350 (the information indicating the detection result, the information for identifying the type of the detection result, and the degree of reliability corresponding to the type of the detection result), the corrected vibration amount acquired from the degree of influence determination unit 350, and a threshold for determining whether or not to adopt the detection result (referred to as the “threshold” hereinafter).

A determination method in the output determination unit 360 includes “a first method of” correcting the degree of reliability using the corrected vibration amount, and comparing the degree of reliability after correction (referred to as the “corrected degree of reliability” hereinafter) with the threshold, to determine whether to adopt or reject the occupant detection result“, “a second method of correcting the threshold using the corrected vibration amount, and comparing the degree of reliability with the corrected threshold, to determine whether to adopt or reject the occupant detection result”, and “a third method of correcting the degree of reliability and the threshold using the corrected vibration amount, and comparing the corrected degree of reliability with the corrected threshold, to determine whether to adopt or reject the occupant detection result”. Any one of these methods may be used.

For example, concretely, the first method is a “method of changing the degree of reliability corresponding to the type of the occupant detection result in accordance with the corrected vibration amount, and, when the changed degree of reliability (corrected degree of reliability) exceeds the threshold, adopting the occupant detection result.”

Further, the second method is a “method of changing the threshold in accordance with the corrected vibration amount, and, when the degree of reliability corresponding to the type of the occupant detection result exceeds the threshold, adopting the occupant detection result.”

Further, the third method is a “method of changing both the degree of reliability corresponding to the type of the occupant detection result, and the threshold in accordance with the corrected vibration amount, and, when the degree of reliability corresponding to the occupant detection result exceeds the threshold, adopting the detection result.”

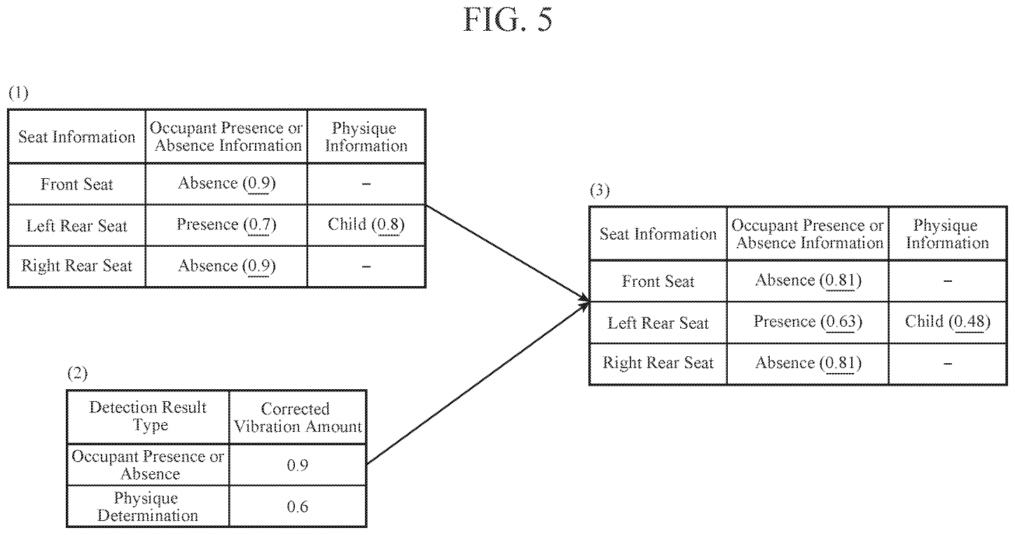

Hereinafter, a concrete example of the first method will be explained using FIG. 5.

FIG. 5 shows tables explaining an example of correcting the degrees of reliability of occupant detection results using the degrees of vibration influence. In FIG. 5, each of the degrees of reliability is shown in ( ).

Assumptions in the explanation will be explained. It is assumed that “(1) the occupant detection results and the degrees of reliability which are outputted by the occupant detection unit 320” and “(2) the corrected vibration amounts calculated by the degree of influence determination unit 350” inputted to the output determination unit 360 are as described in FIG. 5, the threshold of the output determination unit 360 is “0.5”, and the occupant detection unit 320 performs the occupant presence or absence determination and the physique determination. As shown in FIG. 5, for example, the output determination unit 360 multiplies the degrees of reliability in (1) corresponding to the respective types of the detection results by the corrected vibration amounts in (2) corresponding to the respective types of the detection results, thereby changing the degrees of reliability in (2) as shown in (3). Concretely, because the detection result is child and the degree of reliability is 0.8 in the physique determination on the left rear seat in (1), and the corrected vibration amount of the physique determination in (2) is 0.6, the output determination unit 360 changes the degree of reliability corresponding to the result of the physique determination on the left rear seat to 0.48 (=0.8×0.6). Because the degree of reliability of 0.48 does not exceed the threshold of “0.5”, the result of the physique determination on the left rear seat is rejected.

The not-illustrated control unit performs a control process of starting or ending the processing performed in the occupant detection device 300, for example.

The hardware configuration of the occupant detection system 1 will be explained.

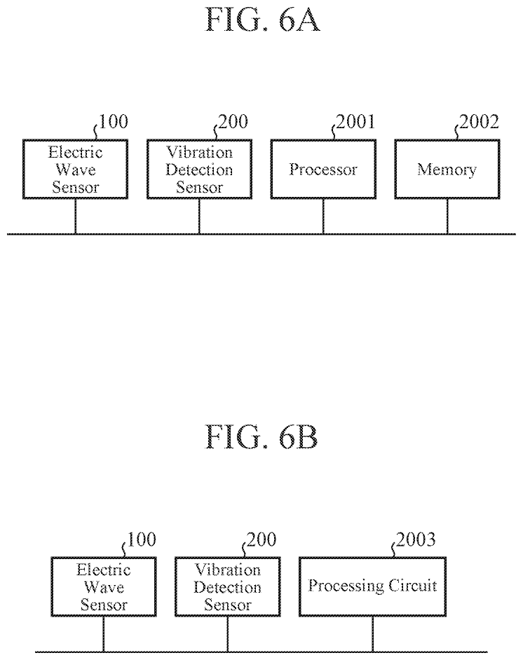

Each of FIGS. 6A and 6B is a diagram showing an example of the hardware configuration of the occupant detection system 1 including the occupant detection device 300.

As shown in FIG. 6A, the occupant detection system 1 is constituted by the electric wave sensor 100, the vibration detection sensor 200, a processor 2001, and a memory 2002. The processor 2001 and the memory 2002 are mounted in, for example, a computer.

In the memory 2002, programs for causing the computer to function as the analysis unit 310, the occupant detection unit 320, the vibration amount calculation unit 330, the normalization processing unit 340, the degree of influence determination unit 350, the output determination unit 360, and the not-illustrated control unit are stored. The functions of the analysis unit 310, the occupant detection unit 320, the vibration amount calculation unit 330, the normalization processing unit 340, the degree of influence determination unit 350, the output determination unit 360, and the not-illustrated control unit are implemented by the processor 2001's reading and executing the programs stored in the memory 2002.

As the processor 2001, for example, a central processing unit (CPU), a graphics processing unit (GPU), a microprocessor, a microcontroller, or a digital signal processor (DSP) is used.

The memory 2002 may be a non-volatile or volatile semiconductor memory such as a random access memory (RAM), a read only memory (ROM), an erasable programmable ROM (EPROM), or a flash memory, a magnetic disc such as a hard disc or a flexible disc, an optical disc such as a compact disc (CD) or a digital versatile disc (DVD), or a magneto-optical disc.

As an alternative, as shown in FIG. 6B, the functions of the analysis unit 310, the occupant detection unit 320, the vibration amount calculation unit 330, the normalization processing unit 340, the degree of influence determination unit 350, the output determination unit 360, and the not-illustrated control unit may be implemented by a processing circuit 2003 for exclusive use. As the processing circuit 2003, for example, a single circuit, a composite circuit, a programmable processor, a parallel programmable processor, an application specific integrated circuit (ASIC), a programmable logic device (PLD), a field-programmable gate array (FPGA), a system-on-a-chip (SoC), or a system large-scale integration (LSI) is used.

As an alternative, a part of the functions of the analysis unit 310, the occupant detection unit 320, the vibration amount calculation unit 330, the normalization processing unit 340, the degree of influence determination unit 350, the output determination unit 360, and the not-illustrated control unit may be implemented by the processor 2001 and the memory 2002, and the remaining functions may be implemented by the processing circuit 2003.

A part of the functions of the analysis unit 310, the occupant detection unit 320, the vibration amount calculation unit 330, the normalization processing unit 340, the degree of influence determination unit 350, the output determination unit 360, and the not-illustrated control unit may be implemented by hardware for exclusive use, and another part of the functions may be implemented by software or firmware. As mentioned above, the processing circuit 2003 in the occupant detection system 1 can implement the above-mentioned functions by using hardware, software, firmware, or a combination of hardware, software and firmware.

Next, the processing performed in the occupant detection device 300 will be explained.

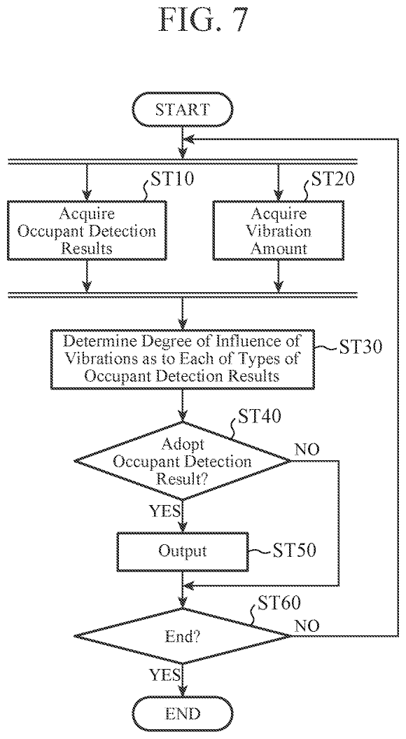

FIG. 7 is a flowchart showing the processing performed in the occupant detection device 300.

The occupant detection device 300 starts the processing at the time when the engine of the vehicle is started, for example.

When receiving a result of analyzing the signals of the electric wave sensor 100 from the analysis unit 310, the occupant detection unit 320 in the occupant detection device 300 performs detection of multiple types including the presence or absence of an occupant, the physique of the occupant, the posture of the occupant, and biological information about the occupant.

The degree of influence determination unit 350 acquires occupant detection results corresponding to the respective types from the occupant detection unit 320 (step ST10).

The degree of influence determination unit 350 in the occupant detection device 300 also acquires the vibration amount of the vehicle via the vibration amount calculation unit 330 and the normalization processing unit 340 (step ST20).

When acquiring occupant detection results and the vibration amount, the degree of influence determination unit 350 uses this vibration amount and the occupant detection results from the occupant detection unit 320, to determine the degree of influence of vibrations as to each of the types of the occupant detection results (step ST30).

When acquiring the degree of influence of vibrations which depends on the type of the occupant detection result from the degree of influence determination unit 350, the output determination unit 360 determines whether or not to adopt the occupant detection result using the degree of influence (step ST40).

When the output determination unit 360 determines not to adopt the occupant detection result (when “NO” in step ST40), the flowchart proceeds to a process of step ST60.

When the output determination unit 360 determines to adopt the occupant detection result (when “YES” in step ST40), the output determination unit 360 causes the occupant detection result to be outputted (step ST50), and the flowchart proceeds to the process of step ST60.

The not-illustrated control unit determines whether or not to end the processing.

When determining to end the processing (when “YES” in step ST60), the not-illustrated control unit causes the processing to be ended, whereas when determining not to end the processing (when “NO” in step ST60), the not-illustrated control unit causes the processing to be repeated.

Amore detailed example of the processing performed in the occupant detection device 300 will be explained using an example of numerical values shown in FIGS. 4 and 5.

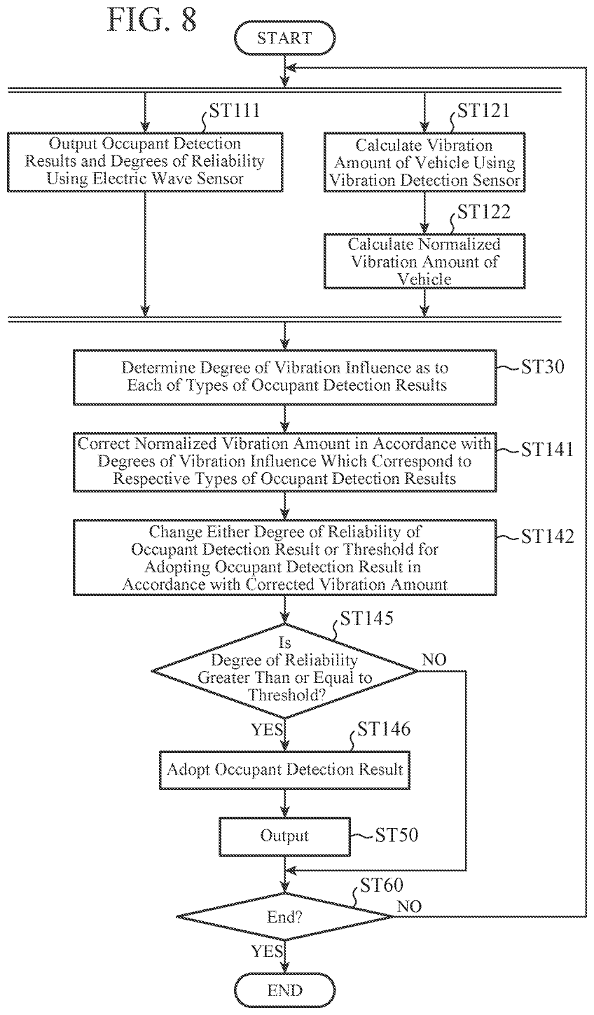

FIG. 8 is a flowchart showing a detailed example of the processing performed in the occupant detection device 300 according to Embodiment 1.

Hereinafter, an example in which it is assumed that the occupant detection unit 320 outputs the presence or absence of an occupant and the physique of the occupant as occupant detection results, and the physique of the occupant, out of the occupant detection results, is erroneously detected will be explained.

It is assumed that the degrees of vibration influence defined in advance as to the respective types of the occupant detection results are “+0.2 for occupant presence or absence” and “−0.1 for physique determination”, as shown in FIG. 4.

Assuming that the threshold used by the output determination unit 360 is “0.5”, and the method of determining the necessity or unnecessity of adopting an occupant detection result in the output determination unit 360 is the first method, the explanation will be made.

The occupant detection unit 320 performs multiple types of occupant detection using the output signals of the electric wave sensor 100, and outputs an occupant detection result about each seat and outputs the degree of reliability as to each of the types of occupant detection results (step ST111). Step ST111 is related to step ST10 in FIG. 7.

Concretely, it is assumed that the occupant detection unit 320 outputs seat information indicating “front seat”, occupant presence or absence information indicating “presence (degree of reliability: 0.7)”, and physique information indicating “child (degree of reliability: 0.8).”

The vibration amount calculation unit 330 calculates the vibration amount of the vehicle using the output signal of the vibration detection sensor 200 (step ST121).

The normalization processing unit 340 normalizes the vibration amount acquired from the vibration amount calculation unit 330, thereby acquiring the normalized vibration amount of “0.7” (step ST122).

Steps ST121 and ST122 are related to step ST20 in FIG. 7.

When acquiring the occupant detection results and the vibration amount, the degree of influence determination unit 350 uses this vibration amount and the occupant detection results from the occupant detection unit 320, to determine the degree of vibration influence as to each of the types of the occupant detection results (step ST30). Concretely, the degree of influence determination unit 350 determines that the degree of vibration influence for the detection result of the occupant presence or absence is “+0.2”, and determines that the degree of vibration influence for the detection result of the physique of the occupant is “−0.1.”

When determining the degrees of vibration influence, the degree of influence determination unit 350 corrects the normalized vibration amount in accordance with the degrees of vibration influence corresponding to the respective types of the occupant detection results (step ST141). Concretely, the degree of influence determination unit 350 adds the degree of vibration influence of “+0.2” for the detection result of the occupant presence or absence to the normalized vibration amount of “0.7”, thereby calculating the corrected vibration amount of “0.9.” The degree of influence determination unit 350 also adds the degree of vibration influence of “−0.1” for the detection result of the physique of the occupant to the normalized vibration amount of “0.7”, thereby calculating the corrected vibration amount of “0.6.”

The output determination unit 360 changes either the degree of reliability of each occupant detection result or the threshold for determining whether to adopt or reject the detection result, in accordance with the vibration amount after the correction (corrected vibration amount) (step ST142). Concretely, in the case of changing the degree of reliability of each occupant detection result, the output determination unit 360 multiplies the degree of reliability of “0.7” of the detection result of the occupant presence or absence and the corrected vibration amount of “0.9” for the detection result of the occupant presence or absence together, thereby calculating the corrected degree of reliability of “0.63.” The output determination unit also multiplies the degree of reliability of “0.8” of the detection result of the physique of the occupant and the degree of vibration influence of “0.6” for the detection result of the physique of the occupant together, thereby calculating the corrected degree of reliability of “0.48.”

The output determination unit 360 determines, for each of the types of the occupant detection results, whether or not the degree of reliability is greater than or equal to the threshold (step ST145). For example, in the case of changing the degree of reliability of each occupant detection result, the output determination unit 360 compares the corrected degree of reliability of “0.63” of the detection result of the occupant presence or absence with the threshold of “0.5”, thereby determining that the corrected degree of reliability is greater than or equal to the threshold.

The output determination unit 360 determines to adopt the detection result of the occupant presence or absence as an occupant detection result (step ST146), when the corresponding one of the degrees of reliability of the respective types of the occupant detection results is greater than or equal to the threshold (when “YES” in step ST145). The output determination unit 360 outputs the occupant seat information indicating “left rear seat” and the occupant presence or absence information indicating “presence” (step ST50).

The output determination unit 360 also compares the corrected degree of reliability of “0.48” of the detection result of the physique of the occupant with the threshold of “0.5”, thereby determining that the corrected degree of reliability is less than the threshold (when “NO” in step ST145). The output determination unit 360 determines that the detection result of the physique of the occupant indicating “child” is erroneous, and rejects the detection result.

Steps ST141, ST142, and ST145 are related to step ST40 in FIG. 7.

Next, the not-illustrated control unit determines whether or not to end the processing.

When determining to end the processing (when “YES” in step ST60), the not-illustrated control unit causes the processing to be ended, whereas when determining not to end the processing (when “NO” in step ST60), the not-illustrated control unit causes the processing to be repeated.

In a state where vibrations occur, the occupant detection device 300 according to Embodiment 1 can output an occupant detection result with a small influence of the vibrations, by taking into consideration the degree of influence which depends on the vibration amount as to each of the types of the occupant detection results. As a result, even in a state where vibrations occur, the occupant detection device 300 can output an occupant detection result with few errors caused by the vibrations.

Further, the occupant detection device 300 according to Embodiment 1 makes it less likely to reduce the degree of reliability of a detection result with a small influence of vibrations, thereby being able to reduce the frequency of erroneous rejection (erroneously rejecting a detection result which is outputted correctly). Further, the degree of reliability of a detection result with a large influence of vibrations is reduced, so that the frequency of erroneous determination can be reduced.

As mentioned above, the occupant detection device according to the present disclosure detects an occupant in a vehicle, and is configured in such a way as to include: the first occupant detection unit to output detection results about the occupant, each of the detection results being detected using an electric wave, each of the detection results being about a corresponding one of detection result types; the degree of influence determination unit to acquire the vibration amount of the vehicle, and to use the vibration amount and the detection results from the first occupant detection unit, to thereby determine, as to each of the detection result types, the degree of influence which depends on the vibration amount; and the output determination unit to determine, using the degree of influence, whether to adopt or reject a corresponding one of the detection results, and to, when determining to adopt the detection result, cause the detection result to be outputted.

As a result, there is provided an advantage of being able to provide an occupant detection device that outputs an occupant detection result with few errors even in a state where vibrations occur.

The occupant detection device according to the present disclosure is further configured in such a way that each of the detection results outputted by the first occupant detection unit includes a degree of reliability, and using the degree of influence, the degree of reliability, and a threshold, the output determination unit determines whether to adopt or reject the detection result.

As a result, the occupant detection device further provides an advantage of being able to output occupant detection results while taking into consideration the degree of reliability of each of the types of the occupant detection results in addition to the degree of influence which depends on the vibration amount.

The occupant detection device according to the present disclosure is further configured in such a way that the output determination unit corrects the degree of reliability using the degree of influence and compares the corrected degree of reliability with the threshold, thereby determining whether to adopt or reject the detection result.

As a result, the occupant detection device further provides an advantage of being able to output occupant detection results while taking into consideration the degree of reliability of each of the types of the occupant detection results in addition to the degree of influence which depends on the vibration amount.

The occupant detection device according to the present disclosure is further configured in such a way that the output determination unit corrects the threshold using the degree of influence and compares the degree of reliability with the corrected threshold, thereby determining whether to adopt or reject the detection result.

As a result, there is further provided an advantage of being able to output occupant detection results while taking into consideration the degree of reliability of each of the types of the occupant detection results in addition to the degree of influence which depends on the vibration amount.

The occupant detection device according to the present disclosure is further configured in such a way that the output determination unit corrects the degree of reliability and the threshold using the degree of influence and compares the corrected degree of reliability with the corrected threshold, thereby determining whether to adopt or reject the detection result.

As a result, there is further provided an advantage of being able to output occupant detection results while taking into consideration the degree of reliability of each of the types of the occupant detection results in addition to the degree of influence which depends on the vibration amount.

The occupant detection method according to the present disclosure is one of detecting an occupant riding in a vehicle, and includes the steps of: by the first occupant detection unit, outputting detection results about the occupant, each of the detection results being detected using an electric wave, each of the detection results being about a corresponding one of detection result types; by the degree of influence determination unit, acquiring the vibration amount of the vehicle, and using the vibration amount and the detection results from the first occupant detection unit, to thereby determine, as to each of the detection result types, the degree of influence which depends on the vibration amount; and by the output determination unit, determining, using the degree of influence, whether to adopt or reject a corresponding one of the detection results, and, when determining to adopt the detection result, causing the detection result to be outputted.

As a result, there is provided an advantage of being able to provide an occupant detection method of outputting an occupant detection result with few errors even in a state where vibrations occur.

### Embodiment 2

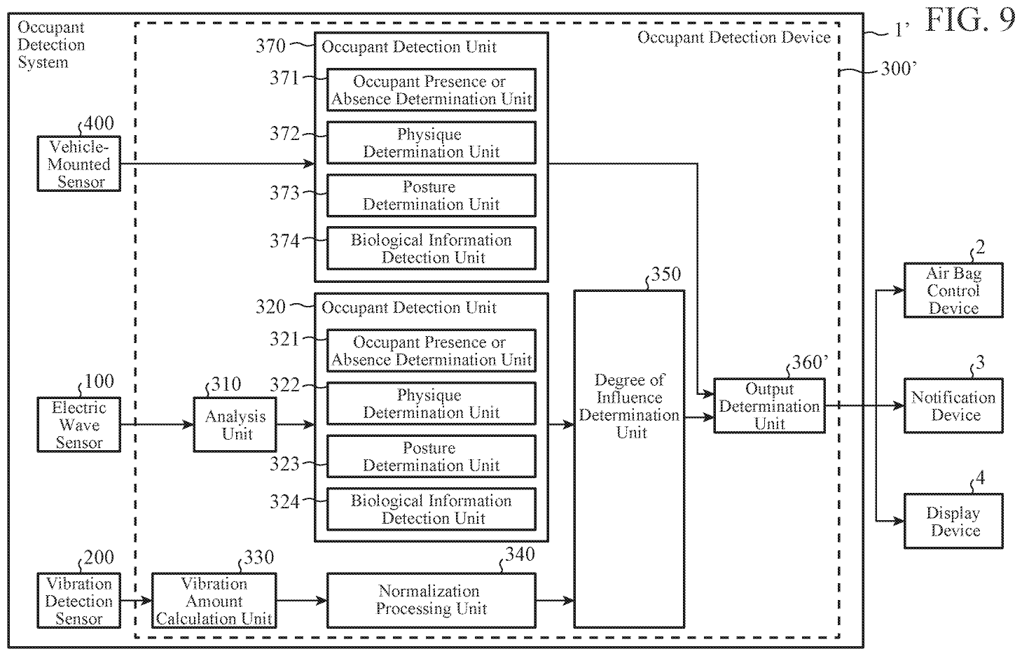

An occupant detection device according to Embodiment 2 and an occupant detection system including this occupant detection device will be explained using FIGS. 9 to 11.

FIG. 9 is a diagram showing the configuration of the occupant detection system including the occupant detection device 300′ according to Embodiment 2.

The occupant detection system 1′ shown in FIG. 9 differs from the occupant detection system 1 shown in FIG. 1 in that a vehicle-mounted sensor 400 is added and the occupant detection device 300 is changed to the occupant detection device 300′.

The occupant detection device 300′ differs from the occupant detection device 300 of FIG. 1 in that an occupant detection unit 370 is added and the output determination unit 360 is changed to an output determination unit 360′.

Hereinafter, an explanation will be made particularly as to the different components using FIG. 9, and an explanation as to the same components as those in FIG. 1 will be omitted as appropriate.

The occupant detection system 1′ shown in FIG. 9 includes an electric wave sensor 100, a vibration detection sensor 200, the vehicle-mounted sensor, and the occupant detection device 300′.

Because the electric wave sensor 100 and the vibration detection sensor 200 are explained in Embodiment 1, a detailed explanation of them will be omitted hereinafter.

The vehicle-mounted sensor detects an occupant by means other than the electric wave sensor 100. The vehicle-mounted sensor is constituted by, for example, a near-infrared camera, a visible light camera, an array microphone, a directional microphone or seating pressure sensor mounted for each seat, or the like. The vehicle-mounted sensor should just be able to acquire information which makes it possible to detect an occupant sitting in each seat.

In the present disclosure, the vehicle-mounted sensor is also referred to as a second sensor that uses means other than an electric wave.

The occupant detection device 300′ shown in FIG. 9 includes, in addition to an analysis unit 310, an occupant detection unit 320, a vibration amount calculation unit 330, a normalization processing unit 340, and a degree of influence determination unit 350, the occupant detection unit 370 and the output determination unit 360′. Further, the occupant detection device 300′ includes a not-illustrated control unit.

The analysis unit 310, the occupant detection unit 320, the vibration amount calculation unit 330, the normalization processing unit 340, the degree of influence determination unit 350, the occupant detection unit 370, and the output determination unit 360′, which are included in the occupant detection device 300′, may be distributed among servers on a network.

Because the analysis unit 310, the occupant detection unit 320, the vibration amount calculation unit 330, the normalization processing unit 340, and the degree of influence determination unit 350 are explained in Embodiment 1, a detailed explanation of them will be omitted.

The occupant detection unit 370 outputs occupant detection results each of which is detected by means other than an electric wave and is about a corresponding one of occupant detection result types.

In the present disclosure, the occupant detection unit 370 is also referred to as a second occupant detection unit. Further, in the present disclosure, the occupant detection results provided by the second occupant detection unit are also referred to as second detection results.

The occupant detection results outputted by the occupant detection unit 370 are, for example, multiple types of detection results: the presence or absence of an occupant of each seat, the physique of the occupant, the posture of the occupant, and biological information about the occupant. Each of the occupant detection results includes, in addition to information indicating the detection result itself, information for identifying the type of the occupant detection result, and a degree of reliability corresponding to the occupant detection result.

Concretely, the occupant detection unit 370 includes at least one of an occupant presence or absence determination unit 371, a physique determination unit 372, a posture determination unit 373, and a biological information detection unit 374.

The occupant presence or absence determination unit 371 outputs, as an occupant detection result detected using a known technique and by means other than an electric wave, information for identifying a seat position, occupant presence or absence information indicating the presence or absence of an occupant, and the degree of reliability of the occupant detection result. The occupant presence or absence information is, for example, binary information indicating presence or absence.

The physique determination unit 372 outputs, as an occupant detection result detected using a known technique and by means other than an electric wave, information for identifying a seat position, physique information indicating the physique of an occupant, and the degree of reliability of the occupant detection result. The physique information is, for example, information indicating a result of classifying the physique of an occupant as one of adult, child and so on.

The posture determination unit 373 outputs, as an occupant detection result detected using a known technique and by means other than an electric wave, information for identifying a seat position, posture information indicating the posture of an occupant, and the degree of reliability of the occupant detection result. The posture information is, for example, information indicating a result of classifying the posture as one of predetermined occupant posture patterns, such as normality, falling sideways, and facedown.

The biological information detection unit 374 outputs, as an occupant detection result detected using a known technique and by means other than an electric wave, information for identifying a seat position, biological information, and the degree of reliability of the occupant detection result. The biological information is, for example, information indicating a breathing rate per minute and a heart rate per minute, or the like.

The occupant detection unit 370 may perform the detection again at predetermined certain intervals or in accordance with situations. The situations include, for example, at least one of a time when the vehicle stops, a time when the vehicle speed becomes less than or equal to a predetermined speed, a time when the vibration amount of the vehicle becomes less than or equal to a predetermined amount, a time when a vehicle door is closed, and a time when a change occurs in the position of an occupant in the vehicle.

The output determination unit 360′ outputs an occupant detection result having a higher degree of reliability, out of the degree of reliability of an occupant detection result detected by means of an electric wave and the degree of reliability of an occupant detection result detected by means other than an electric wave.

Concretely, first, the output determination unit 360′ corrects the degrees of reliability of an occupant detection result provided by the occupant detection unit 320 using a corrected vibration amount. The output determination unit 360′ compares the corrected degree of reliability with the degree of reliability of an occupant detection result provided by the occupant detection unit 370, and thereby selects the occupant detection result having a higher degree of reliability. The output determination unit 360′ compares the degree of reliability of the selected occupant detection result with a threshold, and, when determining that the degree of reliability is greater than or equal to the threshold, causes the occupant detection result to be outputted.

The hardware configuration of the occupant detection system 1′ will be explained.

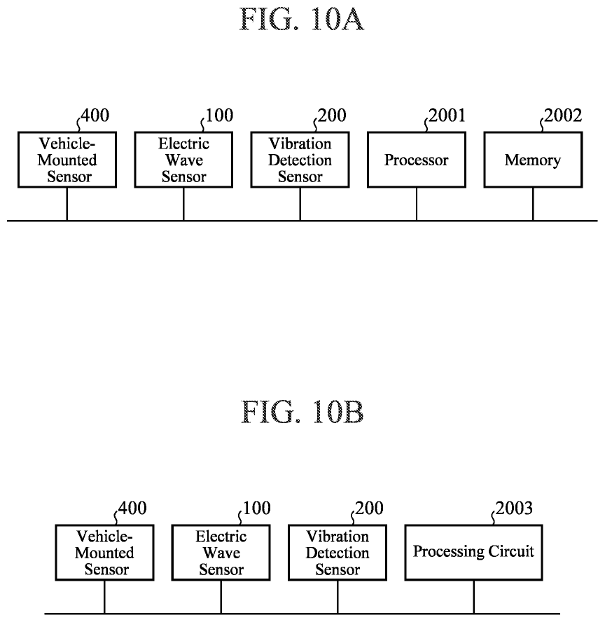

Each of FIGS. 10A and 10B is a diagram showing an example of the hardware configuration of the occupant detection system 1′ including the occupant detection device 300′ according to Embodiment 2.

As shown in FIG. 6A, the occupant detection system 1′ is constituted by the electric wave sensor 100, the vibration detection sensor 200, a processor 2001, and a memory 2002. The processor 2001 and the memory 2002 are mounted in, for example, a computer.

Programs for causing the computer to function as the analysis unit 310, the occupant detection unit 320, the vibration amount calculation unit 330, the normalization processing unit 340, the degree of influence determination unit 350, the occupant detection unit 370, and the output determination unit 360′ are stored in the memory 2002. The functions of the analysis unit 310, the occupant detection unit 320, the vibration amount calculation unit 330, the normalization processing unit 340, the degree of influence determination unit 350, the occupant detection unit 370, and the output determination unit 360′ are implemented by the processor 2001's reading and executing the programs stored in the memory 2002.

The processor 2001 and the memory 2002 are the same as those explained in Embodiment 1.

Further, as shown in FIG. 6B, the functions of the analysis unit 310, the occupant detection unit 320, the vibration amount calculation unit 330, the normalization processing unit 340, the degree of influence determination unit 350, the occupant detection unit 370, and output determination unit 360′ may be implemented by a processing circuit 2003 for exclusive use. As the processing circuit 2003, for example, a single circuit, a composite circuit, a programmable processor, a parallel programmable processor, an application specific integrated circuit (ASIC), a programmable logic device (PLD), a field-programmable gate array (FPGA), a system-on-a-chip (SoC), or a system large-scale integration (LSI) is used.

As an alternative, a part of the functions of the analysis unit 310, the occupant detection unit 320, the vibration amount calculation unit 330, the normalization processing unit 340, the degree of influence determination unit 350, the occupant detection unit 370, and the output determination unit 360′ may be implemented by the processor 2001 and the memory 2002, and the remaining functions may be implemented by the processing circuit 2003.

A part of the functions of the analysis unit 310, the occupant detection unit 320, the vibration amount calculation unit 330, the normalization processing unit 340, the degree of influence determination unit 350, the occupant detection unit 370, and the output determination unit 360′ may be implemented by hardware for exclusive use, and another part of the functions may be implemented by software or firmware. As mentioned above, the processing circuit 2003 in the occupant detection system. 1′ can implement the above-mentioned functions by using hardware, software, firmware, or a combination of hardware, software and firmware.

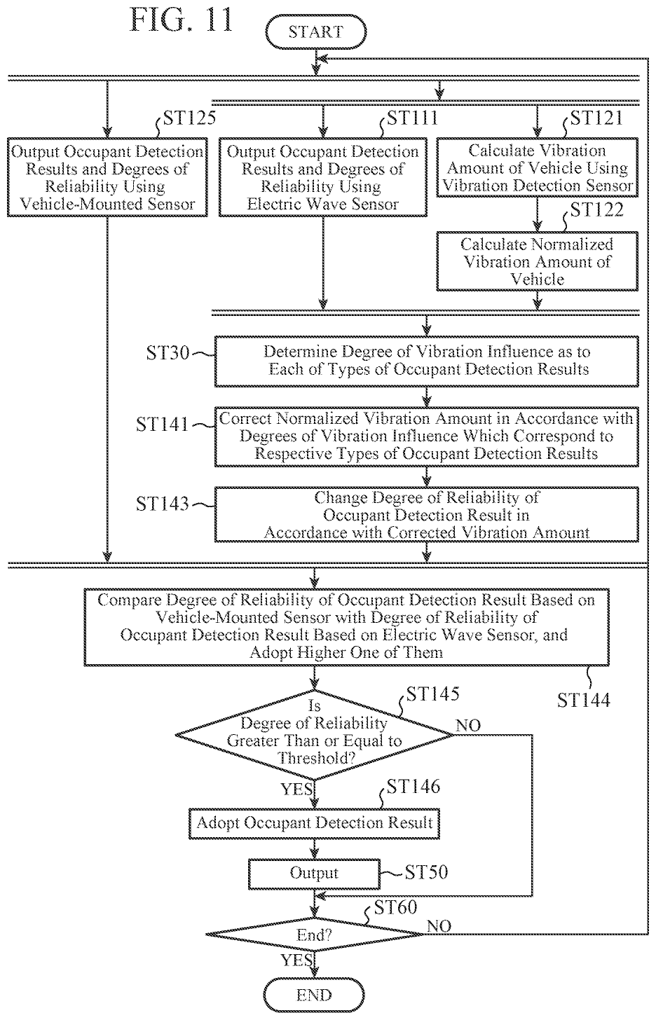

FIG. 11 is a flowchart showing a detailed example of the processing performed in the occupant detection device 300′ according to Embodiment 2.

In the flowchart of FIG. 11, an explanation of the same processes as those in the flowchart of FIG. 8 will be omitted as appropriate.

When starting the processing, the occupant detection device 300′ performs steps ST111, ST121, ST122, ST30, and ST141 which are shown in FIG. 8, and the output determination unit 360′ then changes the degrees of reliability of occupant detection results which are based on the electric wave sensor 100 in accordance with corrected vibration amounts acquired in step ST141 (step ST143).

In parallel to the above-mentioned processes, the occupant detection unit 370 detects an occupant using the vehicle-mounted sensor, and outputs occupant detection results (containing degrees of reliability) (step ST125).

The output determination unit 360′ acquires the occupant detection results from the occupant detection unit 370. The output determination unit 360′ compares the degree of reliability of each occupant detection result which is based on the vehicle-mounted sensor with the degree of reliability, acquired in step ST143, of the corresponding one of the occupant detection results which are based on the electric wave sensor 100, and adopts the occupant detection result having a higher degree of reliability (step ST144).

The output determination unit 360′ determines whether or not the degree of reliability of the occupant detection result is greater than or equal to the threshold (step ST145).

When the degree of reliability of the occupant detection result is greater than or equal to the threshold (when “YES” in step ST145), the output determination unit 360′ adopts the occupant detection result (step ST146).

The output determination unit 360′ causes the adopted occupant detection result to be outputted (step ST50).

Next, the not-illustrated control unit determines whether or not to end the processing.

When determining to end the processing (when “YES” in step ST60), the not-illustrated control unit causes the processing to be ended, whereas when determining not to end the processing (when “NO” in step ST60), the not-illustrated control unit causes the processing to be repeated.

When the degree of reliability of an occupant detection result provided by the electric wave sensor 100 decreases because of vibrations, the occupant detection device 300′ according to Embodiment 2 can adopt an occupant detection result provided the vehicle-mounted sensor which uses means other than an electric wave, and can cause this occupant detection result to be outputted. As a result, even in a state where vibrations occur, the occupant detection device 300′ can output an occupant detection result with few errors caused by the vibrations. More specifically, the robustness of the occupant detection at a time of vibrations of the vehicle is improved.

As mentioned above, the occupant detection device according to the present disclosure is configured in such a way that the occupant detection device includes the second occupant detection unit to output detection results about an occupant with degrees of reliability, each of the detection results being detected using means other than an electric wave, each of the detection results being about a corresponding one of detection result types, and the output determination unit corrects the degree of reliability of a detection result provided by the first occupant detection unit using a degree of influence, and determines whether to adopt or reject a detection result having a higher degree of reliability, out of the corrected degree of reliability and the degree of reliability of a detection result provided by the second occupant detection unit. As a result, there is provided an advantage of being able to provide an occupant detection device that outputs an occupant detection result with few errors even in a state where vibrations occur.

It is to be understood that a free combination of the above-mentioned embodiments can be made, various changes can be made in any component in each of the above-mentioned embodiments, or any component in each of the above-mentioned embodiments can be omitted within the scope of the present disclosure.

### INDUSTRIAL APPLICABILITY

Because the occupant detection device according to the present disclosure can output an occupant detection result with few errors even in a state where vibrations occur, the occupant detection device is suitable for use as an occupant detection device or the like that outputs an occupant detection result to be used for the control of a vehicle.

### REFERENCE SIGNS LIST

1, 1′ occupant detection system, 2 air bag control device, 3 notification device, 4 display device, 100 electric wave sensor (first detection sensor), 200 vibration detection sensor, 300, 300′ occupant detection device, 310 analysis unit, 320 occupant detection unit (first occupant detection unit), 321 occupant presence or absence determination unit, 322 physique determination unit, 323 posture determination unit, 324 biological information detection unit, 330 vibration amount calculation unit, 340 normalization processing unit, 350 degree of influence determination unit, 360, 360′ output determination unit, 370 occupant detection unit (second occupant detection unit), 371 occupant presence or absence determination unit, 372 physique determination unit, 373 posture determination unit, 374 biological information detection unit, 400 vehicle-mounted sensor (second detection sensor), 1001 detection target, 1101 seat information, 1102 occupant presence or absence information, 1103 physique information, 1104 posture information, 1105 biological information, 1201 type of detection result, 1202 degree of influence, 1203 corrected vibration amount (vibration amount after correction), 2001 processor, 2002 memory, and 2003 processing circuit.

1. An occupant detection device that detects an occupant riding in a vehicle, the occupant detection device comprising:
processing circuitry
to output at least one first detection result about the occupant, each of the at least one first detection result being detected using an electric wave, each of the at least one first detection result being about a corresponding one of detection result types;
to acquire a vibration amount of the vehicle, and to use the vibration amount and the at least one first detection result, to thereby determine, as to each of the detection result types, a degree of influence which depends on the vibration amount; and
to determine, using the degree of influence, whether to adopt or reject a corresponding one of the at least one first detection result, and to, when determining to adopt the corresponding one of the at least one first detection result, cause the corresponding one of the at least one first detection result to be outputted.

2. The occupant detection device according to claim 1, wherein
each of the at least one first detection result includes a degree of reliability, and
using the degree of influence, the degree of reliability, and a threshold, the processing circuitry determines whether to adopt or reject the corresponding one of the at least one first detection result.

3. The occupant detection device according to claim 2, wherein the processing circuitry corrects the degree of reliability using the degree of influence and compares the corrected degree of reliability with the threshold, thereby determining whether to adopt or reject the corresponding one of the at least one first detection result.

4. The occupant detection device according to claim 2, wherein the processing circuitry corrects the threshold using the degree of influence and compares the degree of reliability with the corrected threshold, thereby determining whether to adopt or reject the corresponding one of the at least one first detection result.

5. The occupant detection device according to claim 2, wherein the processing circuitry corrects the degree of reliability and the threshold using the degree of influence and compares the corrected degree of reliability with the corrected threshold, thereby determining whether to adopt or reject the corresponding one of the at least one first detection result.

6. The occupant detection device according to claim 2, wherein the at least one first detection result includes at least one of presence or absence of the occupant, physique of the occupant, posture of the occupant, and biological information about the occupant.

7. The occupant detection device according to claim 2, wherein
the processing circuitry outputs at least one second detection result about the occupant with a degree of reliability, each of the at least one second detection result being detected using a means other than an electric wave, each of the at least one second detection result being about a corresponding one of the detection result types, and
the processing circuitry corrects the degree of reliability of the corresponding one of the at least one first detection result provided by the first occupant detection unit using the degree of influence, and determines whether to adopt or reject a detection result having a higher degree of reliability, out of the corrected degree of reliability and the degree of reliability of a corresponding one of the at least one second detection result.

8. The occupant detection device according to claim 7, wherein
the at least one first detection result includes at least one of presence or absence of the occupant, physique of the occupant, posture of the occupant, and biological information about the occupant, and
the at least one second detection result includes the at least one of presence or absence of the occupant, physique of the occupant, posture of the occupant, and biological information about the occupant.

9. An occupant detection method of detecting an occupant riding in a vehicle, the occupant detection method comprising:
outputting at least one first detection result about the occupant, each of the at least one first detection result being detected using an electric wave, each of the at least one first detection result being about a corresponding one of detection result types;
acquiring a vibration amount of the vehicle, and using the vibration amount and the at least one first detection result, to thereby determine, as to each of the detection result types, a degree of influence which depends on the vibration amount; and
determining, using the degree of influence, whether to adopt or reject a corresponding one of the at least one first detection result, and, when determining to adopt the corresponding one of the at least one first detection result, causing the corresponding one of the at least one first detection result to be outputted.
